(19) United States
(12) Patent Application Publication
(10) Pub. No.: US 2008/0114347 A1
(43) Pub. Date: May 15, 2008

(54) Closed Loop Cryosurgical System

(76) Inventors: Douglas A. Devens, Highland Park, IL (US); Steven B. Fine, Minnetonka, MN (US)

Correspondence Address: AMS RESEARCH CORPORATION, 10700 BREN ROAD WEST, MINNETONKA, MN, 55343, US

(21) Appl. No.: 11/939,078

(22) Filed: Nov. 13, 2007

Related U.S. Application Data
(60) Provisional application No. 60/865,582, filed on Nov. 13, 2006.

(51) Int. Cl. A61B 18/02

(52) U.S. Cl. 606/23

(57) ABSTRACT

An improved closed loop cryosurgical system and related methods of use. The closed loop cryosurgical system can utilize a reduced size cryoprobe by moving a pre-cool heat exchanger into a control console for the system. The cyroprobe can further include a sheath having at least one thermal wire spaced around the sheath that can be heated using the control console so as to thaw frozen tissue. By incorporating thermal wiring into the sheath, the time necessary for a cryosurgical procedure can be significantly reduced, especially where multiple freeze-thaw cycles are conducted. In another aspect, an improved, environmentally friendly mixed gas refrigerant can be provided for use in cooling the cyroprobes. The mixed gas refrigerant avoids the use of the refrigerant R22 while also providing faster and more efficient ice ball formation at the cryoprobe tip, therefore allowing the cryosurgical treatment to be accomplished in less time.

### PRIORITY CLAIM

The present application claims priority to U.S. Provisional Application Ser. No. 60/865,582, filed Nov. 13, 2006 and entitled “CLOSED LOOP CRYOSURGICAL SYSTEM”, which is herein incorporated by reference in its entirety.

### FIELD OF THE INVENTION

The present disclosure relates to a cryosurgical system for treatment of benign or cancerous tissues. More particularly, the closed loop cryosurgical system includes design improvement affecting cyroprobe performance so as to speed the process of freezing and thawing tissue as part of a cryosurgical procedure.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used to treat prostate cancer, various types of cancer including renal cancer as well as benign prostate disease. Cryosurgery also has gynecological applications. In addition, cryosurgery may be used for the treatment of a number of other diseases and conditions including breast cancer, liver cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

Cryosurgery often involves a cycle of treatments in which the targeted tissue is frozen, allowed to thaw, and then refrozen. Double and even triple freeze thaw cycles are now commonly used in cryosurgery. Comparison with a single freeze thaw cycle shows that the second freeze thaw cycle will increase damage. This, however, can significantly increase the time it takes to perform a procedure.

### SUMMARY OF THE INVENTION

The present disclosure is directed to a closed loop cryosurgical system and to system advancements for improving the performance of said closed loop cryosurgical system as well as the related methods. Cryosurgical systems can utilize either Joule-Thompson expansion of a refrigerant mixture or a heat exchange fluid to cool cryoprobes for cryosurgery. Once used to cool the cryoprobes, the fluid used in each of the above systems flows back to a control console to be re-cooled and/or re-pressurized.

In one aspect of the present disclosure, a closed loop cryosurgical system utilizing Joule-Thompson expansion has a cryoprobe and/or cryostat heat exchanger module that is reduced in size so as to be more maneuverable during cryosurgical treatment. The size can be reduced by moving a pre-cool heat exchanger, typically located within the cryoprobe handle or the cryostat heat exchanger module, into the control console for the system.

In another aspect of the present disclosure, a sheath for use in a closed loop cryosurgical system can be placed over a cryoprobe. The sheath can be provided with thermal wires that can be used to thaw frozen tissue. Through the use of a sheath having thermal wires, the time necessary for a cryosurgical procedure can be significantly reduced, especially where multiple freeze-thaw cycles are conducted. In some representative embodiments, the thermal wires can be evenly distributed around the sheath to prevent the sheath from overheating and melting during use.

In yet another aspect of the present disclosure, an improved mixed gas refrigerant for use in a cryosurgical system is provided. The mixed gas refrigerant has less environmental impact than most current refrigerants because of its elimination of the refrigerant R22. In addition to its environmental advantages, the mixed gas refrigerant also provides faster and more efficient ice ball formation allowing the cryosurgical treatment to be accomplished in less time.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a side view of an embodiment of a cryosurgical system according to the present disclosure.

FIG. 2A is a side view of a portion of an embodiment of a cryosurgical system according to the present disclosure.

FIG. 2B is a side view of a portion of an embodiment of a cryosurgical system according to the present disclosure.

FIG. 3 is a side view of an embodiment of a cryoprobe sheath according to the present disclosure.

### DETAILED DESCRIPTION

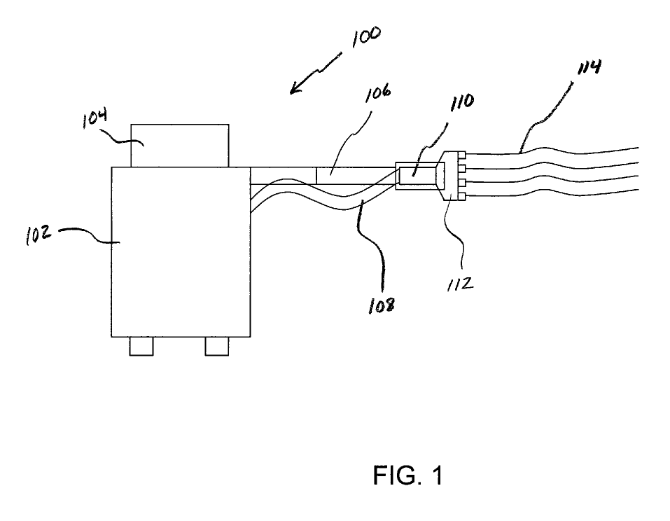

A closed loop cryosurgical system 100 according to the present disclosure is illustrated in FIG. 1. Cryosurgical system 100 can include a refrigeration and control console 102 with an attached display 104. Control console 102 can contain a primary compressor to provide a primary pressurized, mixed gas refrigerant to the system and a secondary compressor to provide a secondary pressurized, mixed gas refrigerant to the system. Control console 102 can also include controls that allow for the activation, deactivation, and modification of various system parameters, such as, for example, the flow rates, pressures, and temperatures of the refrigerants. Display 104 can provide the operator the ability to monitor, and in some embodiments adjust, the system to ensure it is performing properly and can provide real-time display as well as recording and historical displays of system parameters. One exemplary console that can be used with an embodiment of the present invention is used as part of the Her Option® Office Cryoablation Therapy available from American Medical Systems of Minnetonka, Minn.

The high pressure primary refrigerant is transferred from control console 102 to a cryostat heat exchanger module 110 through a flexible line 108. The cryostat heat exchanger module 110 transfers the refrigerant into and receives refrigerant out of one or more cryoprobes 114. The particular cryoprobe configuration will depend on the application for which the system is used. For example, a uterine application will typically use a single, rigid cryoprobe, while a prostate or renal application will use a plurality of flexible cryoprobes as illustrated in the embodiment depicted in FIG. 1. If a single, rigid cryoprobe is used, the elements of the cryostat heat exchanger module 110 may be incorporated into a handle of the cryoprobe. If a plurality of flexible cryoprobes is used, a manifold 112 may be connected to cryostat heat exchanger module 110 to distribute the refrigerant among the several cryoprobes. The cryostat heat exchanger module 110 and cryoprobes 114 can also be connected to the control console 102 by way of an articulating arm 106, which may be manually or automatically used to position the cryostat heat exchanger module 110 and cryoprobes 114. Although depicted as having the flexible line 108 as a separate component from the articulating arm 106, cryosurgical system 100 can incorporate the flexible line 108 within the articulating arm 106.

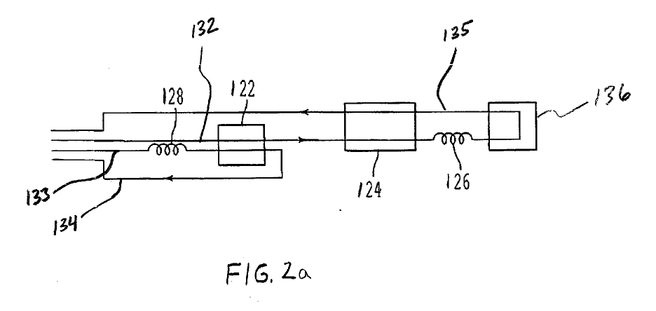

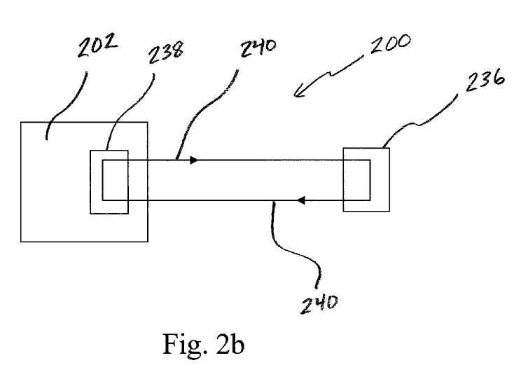

As illustrated in FIG. 2a, cryosurgical system 100 can contain both a pre-cool heat exchanger or pre-cooler 122, and a recuperative heat exchanger or recuperator 124. High pressure primary refrigerant enters the pre-cooler 122 along pathway 132 and is cooled by high pressure secondary refrigerant that enters the pre-cooler 122 and is expanded to a lower temperature by a Joule-Thompson expansion element 128, such as a capillary tube, along pathway 133. The expanded low pressure secondary refrigerant then returns to the secondary compressor within the control console 102 along pathway 134 to be repressurized. The high pressure primary refrigerant then continues into the recuperator 124 where it is further cooled by low pressure primary refrigerant returning from the tip portion 136 of a cryoprobe 114 along pathway 135. The low pressure primary refrigerant is colder than the high pressure primary refrigerant because it has undergone Joule-Thompson expansion in an expansion element 126 in or near the cryoprobe tip portion 136. Tip portion 136 constitutes the region of each cryoprobe 114 that performs the actual cryogenic treatment. The low pressure primary refrigerant then continues along pathway 135 where it returns to the control console 102 to be repressurized.

The precooler 122 can be located within the cryoprobe 114 itself (where only one cryoprobe is used) or within the cryostat heat exchanger module 110 (where multiple cryoprobes are used). However, this tends to require the cryoprobe 114 and/or cryostat heat exchanger module 110 to be relatively large and difficult to maneuver. As an alternative, the precooler 122 can be contained within the control console 102. This can significantly reduce the size and weight of the cryoprobe 114 and/or cryostat heat exchanger module 110, making them much easier to manipulate. An operator can then more easily utilize multiple cryoprobes simultaneously than was previously allowed with the prior bulkier and heavier cryoprobes containing the precooler 122.

As an alternative to the above system utilizing a refrigerant mixture undergoing Joule-Thompson expansion, a cryosurgical system 200 according to the present disclosure, can instead utilize a heat exchange fluid to cool the cryoprobe tips 236, see FIG. 2b. A suitable heat exchange fluid is one that will remain liquid through a broad range of temperatures, ranging at least from the cryosurgical operating temperature to above room temperature. Examples of suitable fluids include Asahi AK-225, Solvay Solexis Galden HT55, and 3M HFE-7200. As illustrated in FIG. 2b, the heat exchange fluid is chilled by a refrigeration circuit 238 in the control console 202 and delivered to the cryoprobe tips 236 through insulated channels 240. As with the previously described cryosurgical system 100, the heat exchange fluid cryosurgical system 200 is preferably a closed loop system. Therefore, once the heat exchange fluid has been used to cool the tip portions 236 of the cryoprobes, the heat exchange fluid flows back to the control console 202 to be re-cooled in the refrigeration circuit 238.

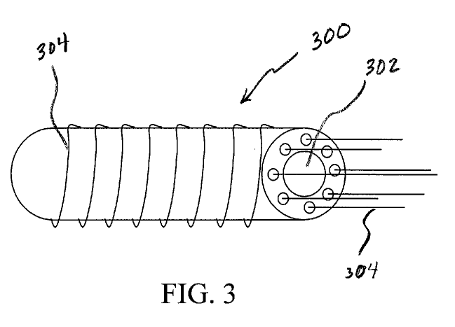

A representative cryoprobe 114 used with the previously discussed cryosurgical systems of the present disclosure can also include a protective sheath 300, as illustrated in FIG. 3. Sheath 300 is positionable over cryoprobe 114 by inserting the cryoprobe tip portion 136 into a sheath opening 302. Sheath 300 is preferably constructed of a thermally resistive material, such as a rigid plastic. Sheath 300 can also include one or more thermal wires 304. In some representative embodiments, thermal wires 304 are evenly distributed around and/or through sheath 300. Thermal wires 304 can comprise a plurality of operably distinct wires or can comprise a single continuous wire wrapped about the sheath 300.

Providing thermal power around the sheath 300 can reduce the time needed to thaw frozen tissue. Where multiple freeze-thaw cycles are employed, this can significantly reduce the time it takes to complete a cryosurgical procedure. Once a targeted area of tissue is frozen, the flow of refrigerant through cryoprobe(s) 114 is stopped using control console 102. Control console 102 is then used to power the thermal wires 304, so that they can be used to thaw the frozen tissue. Once the tissue is sufficiently thawed, thermal power is removed from the thermal wires 304, and the refrigerant is again activated so that the tissue can be re-frozen.

When thermal wire 304 comprises a plurality of distinct thermal wires 304 evenly distributed around sheath 300 the amount of thermal power needed for each wire is small. By limiting the amount of thermal power to each thermal wire 304, the heat density is prevented from being too high in any one area of sheath 300, which prevents the sheath 300 from melting.

Alternatively, when use of a sheath 300 is not desired, thermal wires can be incorporated directly into or around the tip portion 136 of cryoprobe 114. In addition, thermal sensors can be included to measure and control the temperature of the system via the control console 102. Thermal sensors can be located within or on the surface of either a sheath 300 or a cryoprobe 114.

The above described cryosurgical systems preferably use a mixed gas refrigerant. The use of a mixed gas refrigerant is generally known in the art to provide a dramatic increase in cooling performance over the use of a single gas refrigerant. One commonly used gas mixture comprises 50% Krypton, 15% R22, 18% R23, and 17% R116. However, due to recent environmental regulations, as of 2010 the R22 refrigerant (commonly known as “freon”) can no longer be used in new equipment. A more efficient, ozone-friendly gas mixture that can be used as the primary refrigerant in the present system comprises 20-50% Krypton, 0-10% R14, 10-50% R508B, and 0-10% R410A. Preferably, the refrigerant comprises 40% Krypton, 7.5% R14, 48% R508B, and 4.5% R410A. This refrigerant is not only more environmentally friendly than most known refrigerants because of its elimination of R22, but testing has shown that the specific compositions above lead to improved, quicker ice ball formation at tip portion 136 of cryoprobe 114. In addition, most existing cryosurgical systems can be easily adapted to be operated with this refrigerant.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

What is claimed is:

1. A method of performing a cryosurgical procedure, comprising:
activating a control console to circulate refrigerant through one or more cryoprobes, each cryoprobe including a sheath positioned over a cryoprobe tip portion, the sheath have at least one thermal wire wrapped around it;
freezing selected tissue with the cryoprobes;
stopping the flow of refrigerant through the cryoprobes;
powering the at least one thermal wire with the control console to heat the sheath; and
thawing the frozen tissue with the heated sheath.

2. The method of claim 1, further comprising:
removing power from the at least one thermal wire;
recirculating the refrigerant through the cryoprobes; and
refreezing the selected tissue.

3. The method of claim 2, further comprising:
restopping the flow of refrigerant through the cryoprobes;
repowering the at least one thermal wire; and
rethawing the frozen tissue with the at least one thermal wire.

4. The method of claim 1, further comprising:
monitoring the temperatures at the cyroprobe tip portion with thermal sensors and controlling the freezing and thawing temperatures at the cryoprobe tip portion with the console.

5. The method of claim 1, wherein the refrigerant comprises a mixture of 20-50% Krypton, 0-10% R14, 10-50% R508B, and 0-10% R410A by weight of the mixture.

6. The method of claim 5, wherein the mixture comprises 40% Krypton, 7.5% R14, 48% R508B, and 4.5% R410A by weight of the mixture.

7. The method of claim 1, further comprising:
spacing the at least one thermal wire evenly around the sheath to prevent uneven heating of the cyroprobe tip portion.

8. The method of claim 7, wherein the at least one thermal wire comprises a plurality of operably distinct wires.

9. The method of claim 7, wherein the at least one thermal wire comprise a single continuous wire wrapped about the sheath.

10. A closed loop cryosurgical system comprising:
a console having a primary compressor for pressurizing a high pressure primary refrigerant and a secondary compressor for pressurizing a secondary high pressure refrigerant;
a pre-cool heat exchanger configured to expand the secondary high pressure refrigerant and cool the high pressure primary refrigerant with the expanded secondary refrigerant and then return the expanded secondary refrigerant to the secondary compressor to be repressurized;
a cryostat heat exchanger including a recuperative heat exchanger configured to further cool the high pressure primary refrigerant with expanded primary refrigerant;
at least one cryoprobe fluidly connected to the cryostat heat exchanger, the at least one cryoprobe including a tip portion configured to expand the primary refrigerant for performing a cryosurgical procedure; and
a sheath positioned over the at least one cryoprobe, the sheath including a sheath opening into which the tip portion of the cryoprobe is inserted and at least one thermal wire configured to heat the tip portion in order to thaw frozen tissue.

11. The system of claim 10, wherein the at least one thermal wire is evenly distributed around the sheath to prevent uneven heating of the sheath.

12. The system of claim 10, wherein the sheath comprises a thermally resistive material.

13. The system of claim 10, wherein the at least one thermal wire comprises a plurality of operably distinct wires.

14. The system of claim 10, wherein the at least one thermal wire comprises a single continuous wire wrapped about the sheath.

15. The system of claim 10, further comprising a thermal sensor positioned on the sheath, the thermal sensor operably coupled to the console.

16. The system of claim 10, wherein the primary refrigerant and the secondary refrigerant comprises a mixture of 20-50% Krypton, 0-10% R14, 10-50% R508B, and 0-10% R410A by weight of the mixture.

17. A closed loop cryosurgical system comprising:
a console having a primary compressor for pressurizing a high pressure primary refrigerant and a secondary compressor for pressurizing a secondary high pressure refrigerant;
a pre-cool heat exchanger configured to expand the secondary high pressure refrigerant and cool the high pressure primary refrigerant with the expanded secondary refrigerant and then return the expanded secondary refrigerant to the secondary compressor to be repressurized;
a cryostat heat exchanger including a recuperative heat exchanger configured to further cool the high pressure primary refrigerant with expanded primary refrigerant; and
one or more cryoprobes fluidly connected to the cryostat heat exchanger, each cryoprobe including a tip portion configured to expand the primary refrigerant for performing a cryosurgical procedure, wherein the pre-cool heat exchanger is located within the console.

18. The system of claim 17, wherein the system uses a single, rigid cryoprobe and the cryostat heat exchanger is located in a portion of the cryoprobe.

19. The system of claim 17, wherein the system uses a plurality of flexible cryoprobes connected to the system with a manifold connected to the cryostat heat exchanger.

20. The system of claim 10, wherein the primary refrigerant and the secondary refrigerant comprises a mixture of 20-50% Krypton, 0-10% R14, 10-50% R508B, and 0-10% R410A by weight of the mixture.
